(19) United States
(12) Patent Application Publication
(10) Pub. No.: US 2023/0372324 A1
(43) Pub. Date: Nov. 23, 2023

(54) GEL FORMULATIONS COMPRISING MONTELUKAST

(71) Applicant: Diomed Developments Ltd., Loughton (GB)

(72) Inventor: Jasmina Gallagher, Hitchin (GB)

(21) Appl. No.: 18/031,296

(22) PCT Filed: Sep. 30, 2021
(86) PCT No.: PCT/EP2021/077016

§ 371 Date: Apr. 11, 2023

(30) Foreign Application Priority Data
Oct. 13, 2020 (GB) 2016229.3
Jun. 8, 2021 (GB) 2108181.5

(51) Int. Cl. A61K 31/47; A61K 47/02; A61K 47/10; A61K 9/00

(52) U.S. Cl. CPC A61K 31/47; A61K 47/02; A61K 47/10; A61K 9/0014

(57) ABSTRACT

A gel formulation comprising montelukast and a gelling agent for use in the treatment or prevention of eczema and/or atopic dermatitis.

The present invention is directed to a new formulation in the form of a gel comprising montelukast and its use in the treatment and prevention of skin conditions. In particular, a gel formulation comprising montelukast sodium.

### BACKGROUND

Montelukast is used in the treatment of asthma (see for example CN 109528650).

WO 2019/007356 relates to the use of montelukast in combination with mussel adhesive protein in the treatment of inflammation. It has been suggested that montelukast may be useful in the treatment of eczema and atopic dermatitis. However, montelukast has low solubility in water and montelukast salts, such as montelukast sodium, are unstable, and degrades in the presence of light and/or heat and water.

Accordingly, the present invention is directed to compositions which solubilise montelukast sufficiently to enable montelukast to penetrate into the skin.

### SUMMARY

The present disclosure provides a gel formulation comprising montelukast.

The formulation of the present disclosure comprises montelukast and a gelling agent.

In one embodiment, the formulation comprises montelukast, a gelling agent and one or more of a viscosity enhancer, a penetration enhancer, a solubiliser and a solvent. In a further embodiment the composition comprises montelukast, a gelling agent and at least one of each of a viscosity enhancer, a penetration enhancer and a solubiliser. In another embodiment, the composition comprises montelukast, a viscosity enhancer, two or more solubilisers, a solvent and a gelling agent.

In an embodiment, the formulation comprises montelukast sodium, propylene glycol, diethylene glycol ethyl ether (such as transcutol P), propylene glycol monolaurate (such as lauroglycol 90), isoproyl myristate, silica (such as Aerosil 200P) and silicone elastomer (such as ST elastomer 10 HSE). In a further embodiment, the formulation comprises from 0.01 to 15 wt % montelukast sodium, from 2 to 35 wt % propylene glycol, from 2 to 35 wt % diethylene glycol ethyl ether (such as transcutol P), from 2 to 35 wt % propylene glycol monolaurate (such as lauroglycol 90), from 1 to 20 wt % isopropyl myristate, from 1 to 5 wt % silica (such as Aerosil 200 P) and from 10 to 80 wt % silicone elastomer (such as ST Elastomer 10 HSE). In a further embodiment, the formulation comprises from 2 to 10 wt % montelukast sodium, from 5 to 30 wt % propylene glycol, from 5 to 35 wt % diethylene glycol ethyl ether (such as transcutol P), from 5 to 25 wt % propylene glycol monolaurate (such as lauroglycol 90), from 3 to 18 wt % isopropyl myristate, from 1 to 5 wt % silica (such as Aerosil 200 P) and from 10 to 70 wt % silicone elastomer (such as ST Elastomer 10 HSE). In a further embodiment, the formulation comprises from 4 to 6 wt % montelukast sodium, from 10 to 24 wt % propylene glycol, from 10 to 35 wt % diethylene glycol ethyl ether (such as transcutol P), from 7 to 15 wt % propylene glycol monolaurate (such as lauroglycol 90), from 7 to 15 wt % isopropyl myristate, from 1 to 5 wt % silica (such as Aerosil 200 P) and from 10 to 60 wt % silicone elastomer (such as ST Elastomer 10 HSE).

The montelukast used in the present disclosure is montelukast or a pharmaceutically acceptable salt thereof. A particularly preferred form of montelukast is montelukast sodium.

Montelukast is present in the formulation of the present disclosure in an amount of from 0.01 to 15 wt %, preferably from 1 to 12 wt %, more preferably from 2 to 10 wt %, more preferably from 3 to 8 wt %, more preferably 4 to 6 wt % and most preferably about 5 wt %. Montelukast may be present in the form of a salt such as montelukast sodium.

Any gelling agent which forms a gel with montelukast is suitable for use in the present invention. Suitable gelling agents include natural polymers, semisynthetic polymers, synthetic polymers, and inorganic gelling agents. Natural polymers include gelatin, casein, collagen, egg whites, polysaccharides like guar gum, acacia, tragacanth, bug bean gum, pectin, starch, xanthan gum, dextran, succinoglycan. Semisynthetic polymers include cellulose derivatives including carboxy methyl cellulose, ethylcellulose, hydroxyethyl cellulose, hydroxypropyl cellulose, hydroxypropylmethyl cellulose and methylcellulose. Another suitable gelling agent is sodium alginate. A preferred gelling agent is silica.

The gelling agent is present in the formulation of the present disclosure in an amount of from 0.01 to 25 wt %, preferably from 0.5 to 20 wt %, more preferably from 1 to 15 wt %, more preferably from 1 to 5 wt %, more preferably 1.5 to 10% and most preferably about 2 wt % or 2.5 wt %.

Suitable viscosity enhancers include carbomers, sillicas, kolliphors, clays such as magnesium aluminium silicate, waxes and butters. In some embodiments, silicone elastomers are preferred as viscosity enhancers.

The viscosity enhancer may be present in the formulation in an amount of from 10 to 80 wt %, preferably from 10 to 60 wt %, more preferably from 20 to 70 wt %, more preferably from 30 to 70 wt %, most preferably from 50 to 60 wt %.

The penetration enhancer may be selected from borage oil, eucalyptus oil (e.g., Eucalyptus globulus oil, Eucalyptus tereticortis oil, Eucalyptus rostrata), tetrahydropiperine (THP), alcohols (e.g., methanol, ethanol, propanol, octanol, benzyl alcohol, and the like), fatty alcohols (e.g., myristyl alcohol, cetyl alcohol, stearyl alcohol), fatty acids (e.g., oleic acid or decanoic acid), fatty acid esters (e.g., isopropyl myristate, isopropyl palmitate), polyols (e.g., propylene glycol, polyethylene glycol, glycerol), polyethylene glycol monolaurate, lecithin, Spans™, poloxamers, Miglyol™), or combinations thereof. Other suitable penetration enhancers include, diethylene glycol, monoethyl ether (available commercially as Transcutol™), n-decyl methyl sulfoxide, dimethyl sulfoxide, dimethylacetamide, laurocapram (Azone™) dimethylformamide, sucrose monooleate, amides and other nitrogenous compounds (e.g., urea, 2-pyrrolidone, 1-methyl-2-pyrrolidone, ethanolamine, diethanolamine and triethanolamine), terpenes, alkanones, organic acids (e.g., citric acid and succinic acid), terpenes (e.g. cineoles, limonenes) and N-methyl-2-pyrrolidine (Pharmasolve™), or combinations thereof. A particularly preferred penetration enhancer is diethylene glycol monoethyl ether (Transcutol P™).

The penetration enhancer may be present in the formulation in an amount of from 2 to 35 wt %, preferably from 10 to 35 wt %, preferably from 5 to 20 wt %, preferably from 8 to 17 wt %, most preferably from 10 to 15 wt %.

The solubiliser is any substance which increases the solubility of montelukast in the solvent or emollient. The solubiliser may be a glycol or a phospholipid. Particularly preferred solubilisers are propylene glycol and propylene glycol monolaurate.

The solubiliser may be present in the formulation in an amount of from 1 to 50 wt %, preferably 10 to 40 wt %, more preferably from 20 to 30 wt %, most preferably from 7 to 15 wt %.

The solvent is preferably a non-aqueous solvent. Suitable types of solvent include alcohols, glycols, glycol ethers, alkyl esters, glycerides and oils. Suitable alcohols include methanol, ethanol, propanols such as iso-propanol, butanols, benzyl alcohol, other C₄-C₁₀ monoalcohols and mixtures thereof. Suitable glycols include ethylene glycol and propylene glycol. Glycol ethers such as ethylene glycol monomethyl ether, ethylene glycol monoethyl ether, propylene glycol methyl ether, diethylene glycol monomethyl ether and diethylene glycol monoethyl ether may be used. Alkyl esters such as isopropyl myristate, methyl tetradecanoate and ethyl tetradecanoate may be used. Propylene glycol esters such as propylene glycol monolaurate may also be used. DMSO may also be used as a solvent. Solvents that are alcohol free are particularly preferred.

In a preferred embodiment, the formulation is a non-aqueous formulation. In particular the formulation is a non-aqueous formulation that is also alcohol free.

The solvent may be in the form of an emollient. The formulation typically comprises at least one emollient. An emollient helps to smooth and soften the skin, and may also reduce its roughness, cracking or irritation. Non-limiting examples of suitable emollients include, mineral oil having a viscosity in the range of 50 to 500 centipoise (cps), lanolin oil, coconut oil, cocoa butter, olive oil, almond oil, macadamia nut oil, aloe extracts such as aloe Vera lipoquinone, synthetic jojoba oils, natural Sonora jojoba oils, safflower oil, corn oil, liquid lanolin, cottonseed oil and peanut oil. In some embodiments, the emollient is a cocoglyceride, which is a mixture of mono, di and triglycerides of coconut oil or caprylic/capric trigyceride. Other emollients include dicaprylyl ether or a silicone fluid. Suitable emollients may include, squalane, castor oil, polybutene, sweet almond oil, avocado oil, calophyllum oil, ricin oil, olive oil, silicone oils such as dimethylopolysiloxane and cyclomethicone, oleyl alcohol, the oil of cereal germs such as the oil of wheat germ, isopropyl palmitate, octyl palmitate, isopropyl myristate, hexadecyl stearate, butyl stearate, decyl oleate, acetyl glycerides, the octanoates and benzoates of (C12-Cl5) alcohols, the octanoates and decanoates of alcohols and poly alcohols such as those of glycol and glyceryl, ricinoleates esters such as isopropyl adipate, hexyl laurate and octyl dodecanoate, dicaprylyl maleate, hydrogenated vegetable oil, phenyltrimethicone, jojoba oil and aloe vera extract.

Other suitable emollients that are solids or semi solids at ambient temperatures may be used. Such solid or semi-solid cosmetic emollients include, for example, glyceryl dilaurate, hydrogenated lanolin, hydroxylated lanolin, acetylated lanolin, petrolatum, isopropyl lanolate, butyl myristate, cetyl myristate, myristyl myristate, myristyl lactate and cetyl alcohol. Preferably the emollient is butyl myristate, cetyl myristate, isopropyl myristate or myristyl myristate, most preferably isopropyl myristate.

Further suitable emollients include shea butter and castor oil.

The emollient may be present in the formulation in an amount of from 1 to 20 wt %, preferably from 2 to 18 wt %, more preferably from 5 to 15 wt %, more preferably from 7 to 15 wt %, more preferably from 6 to 12 wt %, most preferably about 7 wt %.

The formulation of the present disclosure can be used in the treatment or prevention of skin conditions, in particular atopic dermatitis and eczema. The formulations of the present disclosure are applied topically to the skin.

### EXAMPLES

### Formulation Examples

Formulations were prepared as follows:

Propylene glycol, Transcutol P and Lauroglycol 90 and isopropyl myristate were added to a beaker and mixed thoroughly to form a clear liquid mixture. The mixture was stirred and montelukast sodium was added in small aliquots until fully solubilised. The stirring was continued and Aerosil 200 P was added. Once the mixture appeared uniform, stirring stopped and ST elastomer 10 was added and mixed in using a palette knife.

TABLE 1.1
Prepared Formulations

| | MMM | MMM | MMM | MMM | MMM | MMM | MMM | MMM | MMM | MMM | MMM |
|---|---|---|---|---|---|---|---|---|---|---|---|
| Formulation | 1/2 | 2/1 | 3/1 | 4/1 | 5/1 | 6/1 | 7/1 | 8/2 | 9/1 | 9/2 | 9/3 |
| Propylene | 23.00 | 23.00 | 23.00 | 23.00 | 12.50 | 12.50 | 12.50 | 12.50 | 17.75 | 17.75 | 17.75 |
| glycol EP | | | | | | | | | | | |
| Transcutol P | 29.00 | 29.00 | 15.50 | 15.50 | 29.00 | 29.00 | 15.50 | 15.50 | 22.25 | 22.25 | 22.25 |
| Lauroglycol 90 | 14.00 | 7.50 | 14.00 | 7.50 | 14.00 | 7.50 | 14.00 | 7.50 | 10.75 | 10.75 | 10.75 |
| Isopropyl | 14.00 | 7.50 | 7.50 | 14.00 | 7.50 | 14.00 | 14.00 | 7.50 | 10.75 | 10.75 | 10.75 |
| myristate | | | | | | | | | | | |
| Montelukast | 5.19 | 5.19 | 5.19 | 5.19 | 5.19 | 5.19 | 5.19 | 5.19 | 5.19 | 5.19 | 5.19 |
| sodium EP | | | | | | | | | | | |
| Aerosil 200 P | 2.00 | 2.00 | 2.00 | 2.00 | 2.00 | 2.00 | 2.00 | 2.00 | 2.00 | 2.00 | 2.00 |
| ST Elastomer | 12.81 | 25.81 | 32.81 | 32.81 | 29.81 | 29.81 | 36.81 | 49.81 | 31.31 | 31.31 | 31.31 |
| 10 HSE | | | | | | | | | | | |

### Test Example

To assess the percutaneous absorption and distribution of sodium montelukast from the formulations above, each formulation was applied to ex vivo full thickness human skin that has been tape stripped to mimic the impaired skin barrier function characteristic of atopic dermatitis (AD) and absorption and distribution of montelukast monitored over 72 hours.

Method

All eleven formulations were tested simultaneously on human skin from three donors.

Three skin replicates (n=3) were tested on each formulation.

After thawing and removal of the fat tissue, the stratum corneum of each skin replicate was impaired using tape stripping procedure. Tape stripping involved consecutively applying and removing ten tape strips (D101—D-Squame Standard Sampling Discs, diameter 14 mm) to the surface of each skin sample under constant pressure using index finger.

The skin samples were mounted in Franz diffusion cells (unjacketed) with 1 cm² test area, 2 mL receptor volume, and flat ground joint) in a temperature-controlled water bath (to ensure that the skin surface temperature was maintained at 32° C.) with continuous magnetic stirring in the receptor compartments (250 rpm). Care was taken to ensure that the tape stripped area was located at the centre of Franz cells. Skin samples were left to equilibrate for one hour prior to applying the test formulations. The formulations were weighed and applied carefully under the aluminium foil using a glass rod and spread across the skin. The glass rods were weighed before and after use to determine the exact amount of each formulation being applied.

At pre-established time intervals after application (6 h, 24 h, 48 h and 72 h) 100 μL aliquots were withdrawn from the receptor phases and replaced with an equal volume of fresh preheated phosphate buffer solution (PBS) (32° C.) containing 50% v/v HPLC grade ethanol. Withdrawn samples were stored in the fridge (4° C.) and subjected by HPLC analysis within 72 h.

Positive controls (PC, three replicates for each formulation) were also prepared as follows: approximately 5 mg formulation were added to 10 mL PBS containing 50% v/v HPLC grade ethanol in glass vials wrapped in aluminium foil and left for 72 h in the same experimental conditions. The exact amount of each formulation was recorded.

After the 72 h measurement was completed, the skin samples were taken out of Franz cells, placed on filter papers and covered by a sheet of aluminium foil. The stratum corneum of each skin replicate was wiped thoroughly with three double-ended cotton buds to remove any surplus formulation remaining on the skin (the same technique was used for all samples). After that, ten tape strips were applied consecutively to the surface of each skin sample (under constant finger pressure) and removed, and the active ingredient extracted from the tape strips by immersing them in 1 mL PBS containing 50% v/v HPLC grade ethanol over the weekend. Tape stripping procedure was performed whilst skin replicates were placed under a sheet of aluminium foil, to ensure minimal exposure to UV light.

Once each experiment had been completed and the sample batch was ready for HPLC analysis, fresh positive controls (three replicates for each formulation) were also prepared as follows: approximately 5 mg of montelukast sodium were added to 10 mL PBS containing 50% v/v HPLC grade ethanol in glass vials wrapped in aluminium foil, the vials were sonicated for 20 min, the solutions was filtered through PTFE 0.45 μm filters in Amber glass vials and subjected to HPLC analysis.

Tape stripping was used to mimic the compromised skin barrier function that is characteristic of atopic dermatitis skin, and thereby allow these experiments to measure the percutaneous absorption and distribution of the test formulations under conditions more representative of the normal clinical situation.

In this study, 10 tape strips were used to assess the amount of drug left in the stratum corneum at the completion of the study after the surplus of formulation left on the skin had been removed with cotton buds.

### Results and Discussion

Penetration Profile Through Tape Stripped Skin

Sodium montelukast was detected and quantified to have permeated through all thickness of tape stripped skin for one formulation (MMM6/1) at 72 h in Experiment No. 1 (5.272 μg/cm² which is 1.517% of applied dose).

Sodium Montelukast in the Stratum Corneum

The average amount of drug that was extracted from stratum corneum (Mean±SD, n=3 skin donors) following 72 h exposure ranged between 2.007±0.762 μg/cm² (MMM8/2, which is 0.700±0.195% of applied dose) to 3.929±1.187 μg/cm² (MMM9/1, which is 1.082±0.282% of applied dose)—see Table 1.2 and Table 2.

Low levels of sodium montelukast were expected to be quantified in the stratum corneum as the skin was already tape stripped prior to experiment taking place, and the available stratum corneum was only few layers thick.

Sodium Montelukast on the Surface of the Skin

The average amount of sodium montelukast recovered from cotton buds following 72 h exposure ranged between 211.833±57.019 μg/cm² (Mix MMM6/1) to 284.870±37.812 μg/cm² (MMM4/1)—see Table 1, or between 61.597±16.759% of applied dose (MMM6/1) to 92.497±32.659% of applied dose (MMM2/1)—see Table 2.

Summary Data

Table 1.2 and Table 2 summarise the data for the amount and percentage of sodium montelukast permeated through tape stripped human skin over 72 hours post-application, and the amount of drug recovered from tape strips and from cotton.

TABLE 1.2
Absorption parameters over 72 h on tape stripped human skin ex vivo
following single applications of eleven formulations containing 5.19% sodium
montelukast (μg/cm², Mean ± SD, n = 3, 3 skin donors).

| | | | | Left on the skin: |
|---|---|---|---|---|
| | Starting point: | | Penetrated the stratum | amount of drug |
| | amount sodium | | corneum: amount of | recovered from |
| | montelukast applied | Penetrated through | drug recovered from 10 | cotton buds |
| | (μg/cm²) | the skin (μg/cm²) | tape strips (μg/cm²) | (μg/cm²) |
| Formulation | Mean (n = 3) | Mean (n = 3) | Mean (n = 3) | Mean (n = 3) |
| MMM1/2 | 316.590 ± 35.957 | 0.000 ± 0.000 | 2.436 ± 1.718 | 221.920 ± 70.922 |
| MMM2/1 | 316.590 ± 58.488 | 0.000 ± 0.000 | 2.944 ± 2.761* | 280.243 ± 41.841* |
| MMM3/1 | 344.270 ± 60.153 | 0.000 ± 0.000 | 2.762 ± 1.135 | 264.027 ± 73.796 |
| MMM4/1 | 404.820 ± 23.784 | 0.000 ± 0.000 | 3.749 ± 1.194 | 284.870 ± 37.812 |
| MMM5/1 | 318.320 ± 40.313 | 0.000 ± 0.000 | 3.118 ± 0.845 | 276.317 ± 53.094 |
| MMM6/1 | 344.270 ± 5.993 | 1.757 ± 3.044″″ | 3.342 ± 0.434** | 211.833 ± 57.019 |
| MMM7/1 | 318.320 ± 26.633 | 0.000 ± 0.000 | 2.791 ± 0.708*** | 253.767 ± 14.541 |
| MMM8/2 | 288.910 ± 92.308 | 0.000 ± 0.000 | 2.007 ± 0.762 | 261.980 ± 99.590 |
| MMM9/1 | 359.840 ± 20.975 | 0.000 ± 0.000 | 3.929 ± 1.187 | 269.017 ± 38.302 |
| MMM9/2 | 368.490 ± 8.989 | 0.000 ± 0.000 | 3.513 ± 0.692″ | 245.087 ± 64.488 |
| MMM9/3 | 354.650 ± 19.649 | 0.000 ± 0.000 | 3.189 ± 1.164 | 253.367 ± 67.786 |

*MMM2/1 - cotton buds (experiment No. 3): stratum corneum detached entirely while swapping the skin surface with cotton buds
**MMM6/1 - tape strips (experiment No. 3): stratum corneum detached entirely while tape stripping
***MMM7/1 - tape strips (experiment No. 3): stratum corneum detached entirely while tape stripping
″MMM9/2 - tape strips (experiment No. 3): stratum corneum detached entirely while tape stripping
″″MMM6/1 - replicate MMM6/1-72 h was the only replicate in which sodium montelukast was detected and quantified to have penetrated through the all thickness of tape stripped skin at 72 h (Experiment No. 1).

TABLE 2
Absorption parameters over 72 h on tape stripped human skin ex vivo following
single applications of eleven formulations containing 5.19% w/w sodium montelukast
(% of applied dose/cm², Mean ± SD, n = 3, 3 skin donors).

| | | Penetrated the | | |
|---|---|---|---|---|
| | | stratum corneum: | Left on the skin: | |
| | | amount of drug | amount of drug | |
| | Penetrated through | recovered from 10 tape | recovered from | |
| | the skin (%/cm²) | strips (%/cm²) | cotton buds (%/cm²) | Total % |
| Formulation | Mean (n = 3) | Mean (n = 3) | Mean (n = 3) | Mean (n = 3) |
| MMM1/2 | 0.000 ± 0.000 | 0.771 ± 0.506 | 69.019 ± 15.842 | 69.790 ± 15.607 |
| MMM2/1 | 0.000 ± 0.000 | 0.839 ± 0.783* | 92.497 ± 32.659 | 93.336 ± 31.890 |
| MMM3/1 | 0.000 ± 0.000 | 0.827 ± 0.358 | 75.679 ± 8.982 | 76.506 ± 8.855 |
| MMM4/1 | 0.000 ± 0.000 | 0.933 ± 0.333 | 70.710 ± 11.703 | 71.644 ± 11.660 |
| MMM5/1 | 0.000 ± 0.000 | 0.975 ± 0.228 | 88.746 ± 26.495 | 89.721 ± 26.341 |
| MMM6/1 | 0.505 ± 0.875″″ | 0.970 ± 0.114** | 61.597 ± 16.759 | 63.072 ± 15.952 |
| MMM7/1 | 0.000 ± 0.000 | 0.882 ± 0.253*** | 79.835 ± 2.050 | 80.717 ± 2.164 |
| MMM8/2 | 0.000 ± 0.000 | 0.700 ± 0.195 | 89.639 ± 5.048 | 90.338 ± 5.078 |
| MMM9/1 | 0.000 ± 0.000 | 1.082 ± 0.282 | 75.256 ± 14.140 | 76.339 ± 13.976 |
| MMM9/2 | 0.000 ± 0.000 | 0.956 ± 0.203″ | 66.745 ± 18.747 | 67.701 ± 18.949 |
| MMM9/3 | 0.000 ± 0.000 | 0.914 ± 0.389 | 71.126 ± 16.419 | 72.039 ± 16.193 |

*MMM2/1 - cotton buds (experiment No. 3): stratum corneum detached entirely while swapping the skin surface with cotton buds
**MMM6/1 - tape strips (experiment No. 3): stratum corneum detached entirely while tape stripping
***MMM7/1 - tape strips (experiment No. 3): stratum corneum detached entirely while tape stripping
″MMM9/2 - tape strips (experiment No. 3): stratum corneum detached entirely while tape stripping
″″MMM6/1 - replicate MMM6/1-72 h was the only replicate in which sodium montelukast was detected and quantified to have penetrated through the all thickness of tape stripped skin at 72 h (Experiment No. 1).

In order to check the analysis of montelukast, positive controls containing montelukast in a buffer were analysed after keeping for 72 hours and fresh positive controls of montelukast in a buffer were analysed immediately. Both showed that montelukast was detected at expected levels.

In this ex vivo study, similar amounts of drug were detected and quantified in stratum corneum for all eleven tested formulations. At 72 hours post-application sodium montelukast could be detected and quantified to have penetrated through all thicknesses of tape stripped skin for only one tested replicate (MMM6/1—Experiment No. 1.)

1. A gel formulation comprising montelukast and a gelling agent.

2. A gel formulation according to claim 1 further comprising one or more of a viscosity enhancer, a penetration enhancer, a solubiliser and a solvent.

3. A gel composition according to claim 2 wherein the formulation comprises at least one of each of a viscosity enhancer, a penetration enhancer and a solubiliser.

4. A gel formulation according to claim 1, wherein the formulation is a non-aqueous formulation.

5. A gel formulation according to claim 1, wherein the formulation is alcohol free.

6. A gel formulation according to claim 1, wherein the composition comprises montelukast, a viscosity enhancer, two solubilisers, a solvent and a gelling agent.

7. A gel formulation according to claim 6 wherein the formulation comprises montelukast sodium, silicone elastomer, diethylene glycol ethyl ether, propylene glycol, propylene glycol monolaurate, isopropyl myristate and anhydrous colloidal silica.

8. A gel formulation according to claim 1, wherein the amount of montelukast is from 0.01 to 15 wt %.

9. A gel formulation according to claim 1, wherein the amount of montelukast is from 3 to 10 wt %.

10. A gel formulation according to claim 1, wherein the montelukast is montelukast sodium.

11. A gel formulation according to claim 1, where the formulation comprises from 4 to 6 wt % montelukast sodium, from 10 to 24 wt % propylene glycol, from 10 to 35 wt % diethylene glycol ethyl ether, from 7 to 15 wt % propylene glycol monolaurate, from 7 to 15 wt % isopropyl myristate, from 1 to 5 wt % anhydrous colloidal silica and from 10 to 60 wt % silicone elastomer.

12. The gel formulation according to claim 11, for use in the treatment or prevention of eczema and/or atopic dermatitis.
